(19) United States
(12) Patent Application Publication
(10) Pub. No.: US 2025/0236570 A1
(43) Pub. Date: Jul. 24, 2025

(54) ONE-STEP PROCESS OF MAKING BCI

(71) Applicant: INTERNATIONAL FLAVORS & FRAGRANCES INC., New York, NY (US)

(72) Inventors: Carlos LOPEZ-CRUZ, Benicarlo (ES); Jordi PASTOR-FOLCH, Benicarlo (ES)

(21) Appl. No.: 18/853,664

(22) PCT Filed: Mar. 31, 2023
(86) PCT No.: PCT/US2023/017132

§ 371 Date: Oct. 2, 2024

(30) Foreign Application Priority Data
Apr. 6, 2022 (EP) 22382326.1

(51) Int. Cl. C07C 1/22

(52) U.S. Cl. CPC C07C 1/22; C07C 2521/04; C07C 2521/08; C07C 2523/42; C07C 2523/44; C07C 2602/32

(57) ABSTRACT

This disclosure relates to a one-step process for making BCI. The process involves contacting a starting material comprising MCDDK with hydrogen in the presence of a catalyst mixture in a reaction zone to produce a product mixture comprising BCI, wherein the catalyst mixture comprises a solid acid catalyst and a hydrogenation catalyst.

### BACKGROUND

### Field of the Disclosure

The present disclosure relates to a one-step process for making BCI by using MCDDK as the starting material. More particularly the process uses a catalyst mixture comprising a solid acid catalyst and a hydrogenation catalyst.

### Description of Related Art

Muscenone is one of the most powerful commercially available macrocyclic musks. It is a very good, versatile musk with some resemblance to musk ketone and Muscone in its odour and applications in flavour and fragrances. Saturated macrocyclic ketones having 14- to 18-membered rings, e.g. muscone (3-methylcyclopentadecanone), are sought-after fragrances or flavors. Since the compounds from natural sources are available only in small amounts, the synthesis of these has been the subject matter of comprehensive studies. All methodologies currently developed involves multi-step synthesis with a limited overall performance respect the starting material.

One industrial efficient route of making muscenone starts from MCDDK and consists of a 6-steps synthesis involving the important intermediate product BCI. BCI is formed from MCDDK (obtained from CDDK) through a 3-steps synthetic route involving dehydrocyclization towards the corresponding multi-unsaturated bicyclic compound, hydrogenation towards the mono-unsaturated bycyclic compounds (e.g., BCI and its position isomers) and isomerization towards BCI. Disadvantages of current methodology are the limited overall yield to produce BCI from MCDDK (58-63 wt % respect the starting material), the need to employ at least a set of three different operation units (one of them being an autoclave) and the amount of waste produced.

### BRIEF SUMMARY OF THE DISCLOSURE

The present disclosure provides a one-step process for making BCI. The one-step process comprises contacting a starting material comprising MCDDK with hydrogen in the presence of a catalyst mixture in a reaction zone to produce a product mixture comprising BCI, wherein the catalyst mixture comprises a solid acid catalyst and a hydrogenation catalyst.

### DETAILED DESCRIPTION

The foregoing general description and the following detailed description are exemplary and explanatory only and are not restrictive of the invention, as defined in the appended claims. Other features and benefits of any one or more of the embodiments will be apparent from the following detailed description, and from the claims.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8”, “3 to 10”, “2 to 7”, “1.5 to 6”, “3.4 to 7.8”, “1 to 2 and 7-10”, “2 to 4 and 6 to 9”, “1 to 3.6 and 7.2 to 8.9”, “1-5 and 10”, “2 and 8 to 10”, “1.5-4 and 8”, and the like.

While compositions and methods are described herein in terms of “comprising” various components or steps, the compositions and methods also can “consist essentially of” or “consist of” the various components or steps, unless stated otherwise.

Some alkene molecules may exist as cis or trans stereoisomers. Unless explicitly indicated, an alkene (molecule, structure, formula, or chemical name) as used herein includes both cis and trans stereoisomers, as well as any combinations or mixtures of the cis and trans stereoisomers.

Before addressing details of embodiments described below, some terms are defined or clarified.

The term “MCDDK”, as used herein, is the chemical compound named 2-(2-methyl-2-propenyl)cyclododecan-1-one, 2-(2-methylprop-2-enyl)cyclododecan-1-one or 2-(2-methylallyl)cyclododecan-1-one and represented by the following Formula (I):

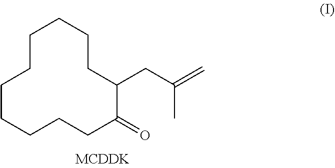 [SMILES: C=C(C)CC1CCCCCCCCCCC1=O]

The term “BCI”, as used herein, is the chemical compound named 14-methylbicyclo[10.3.0]pentadec-[1,(12)]-ene or 2,3,4,5,6,7,8,9,10,11,12,13-dodecahydro-2-methyl-1H-cyclopentacyclododecene and represented by the following Formula (II):

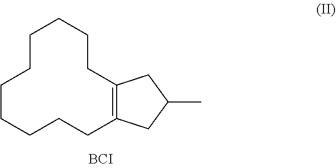 [SMILES: CC1CC2=C(CCCCCCCCCC2)C1]

The term “MDIENE”, as used herein, is the chemical compound named 14-methylbicyclo[10.3.0]pentadec-[1,(12)]-14-diene and represented by the following Formula (III).

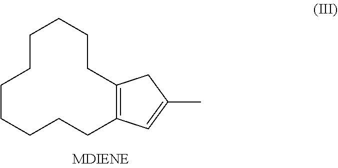 [SMILES: CC1=CC2=C(CCCCCCCCCC2)C1]

The term “solid acid catalyst”, as used herein, means a solid catalyst comprising Brønsted acid (or protic) sites and/or Lewis acid (aprotic) sites. Brønsted acid site is a site with an ionizable hydrogen atom. Lewis acid site is an electron accepting site.

The term “amorphous”, as used herein, means a solid without crystalline ordered structure.

The term “fixed-bed catalyst”, as used herein, means a catalyst, typically in pellet or granule form, packed in a static bed that allows a gas or liquid to pass through.

The term “contact time” or “reaction time”, as used herein, is calculated by dividing the weight of the catalyst mixture (in grams) present in the reaction zone by the MCDDK flow rate through the reaction zone. The MCDDK flow rate is expressed as the weight amount of MCDDK (in grams) passing through the reaction zone per hour. This calculation is particularly applicable to a process conducted in continuous mode in a reaction zone with fixed-bed catalysts. The terms “contact time” and “reaction time” can be used interchangeably in this disclosure.

The term “yield of BCI”, as used herein, means the total molar amount of BCI produced in the process of this disclosure comparing with the total molar amount of MCDDK (limiting reactant).

The present disclosure provides a one-step process for making BCI. The one-step process comprises contacting a starting material comprising MCDDK with hydrogen in the presence of a catalyst mixture in a reaction zone to produce a product mixture comprising BCI, wherein the catalyst mixture comprises a solid acid catalyst and a hydrogenation catalyst.

In some embodiments, the starting material comprises at least 80 wt %, or at least 85 wt %, or at least 90 wt %, or at least 95 wt %, or at least 98 wt %, or at least 99 wt % of MCDDK based on the total weight of the starting material. In some embodiments, the starting material consists essentially of or consists of MCDDK.

In some embodiments, no more than 10 wt %, or no more than 5 wt %, or no more than 2 wt %, or no more than 1 wt %, or no more than 0.5 wt %, or no more than 0.2 wt %, or no more than 0.1 wt % of basic compound(s), such as amine and/or nitro compound(s), are present in the reaction zone based on the total weight of the starting material. In some embodiments, the reaction zone is substantially free or free of any basic compounds. In some embodiments, no more than 10 wt %, or no more than 5 wt %, or no more than 2 wt %, or no more than 1 wt %, or no more than 0.5 wt %, or no more than 0.2 wt %, or no more than 0.1 wt % of water is fed into the reaction zone based on the total weight of the starting material. This includes water carried by the starting material and hydrogen. In some embodiments, substantially no or no water is fed into the reaction zone. In some embodiments, no more than 10 wt %, or no more than 5 wt %, or no more than 2 wt %, or no more than 1 wt %, or no more than 0.5 wt %, or no more than 0.2 wt %, or no more than 0.1 wt % of sulfur-containing compound(s) are present in the reaction zone based on the total weight of the starting material. In some embodiments, the reaction zone is substantially free or free of any sulfur-containing compounds.

MCDDK and hydrogen can be fed separately or together into the reaction zone. In some embodiments, MCDDK and hydrogen are mixed and fed together into the reaction zone. In some embodiments, MCDDK is pre-heated and vaporized to form MCDDK gas to be fed into the reaction zone. Typically, the mole ratio of hydrogen to MCDDK fed into the reaction zone is from about 5:1 to about 100:1, or from about 10:1 to about 100:1, or from about 10:1 to about 60:1, or from about 20:1 to about 40:1. In some embodiments, the upper limit of the mole ratio is 100, 90, 80, 70, 60, 50, 40, 30, or 20. In some embodiments, the lower limit of the mole ratio is 1, 2, 3, 4, 5, 6, 7, 8, 9, 10, 11, 12, 13, 14, 15, 16, 17, 18, 19, or 20.

In some embodiments, the process of this disclosure is conducted in substantial absence or absence of a solvent. In some embodiments, MCDDK and hydrogen are present in the reaction zone in gaseous state, that is, MCDDK gas and hydrogen gas are contacted in the presence of the catalyst mixture in the reaction zone. The catalyst mixture comprises a solid acid catalyst and a hydrogenation catalyst. In some embodiments, the reaction zone is a fixed bed reactor and may be of a plug flow, tubular or other design packed with solid catalysts (i.e. a packed bed reactor), that is, the solid acid catalyst and the hydrogenation catalyst are present in the reaction zone as fixed-bed catalysts. The process of this disclosure can be conducted in batch or continuous mode. In some embodiments, the process is conducted in continuous mode. For example, MCDDK gas and hydrogen gas can be continuously fed into the reaction zone, pass through the fixed-bed catalysts and emerge as a continuous stream of product mixture.

In some embodiments, the solid acid catalyst is selected from the group consisting of alumina, silica-alumina, zeolite, silico-alumino-phosphate, aluminophosphate, sulfated zirconia, zirconia (zirconium dioxide), zinc oxide, and mixtures thereof. In some embodiments, the solid acid catalyst is selected from the group consisting of alumina, silica-alumina, zeolite, sulfated zirconia, and mixtures thereof. In some embodiments, the solid acid catalyst is selected from the group consisting of alumina, silica-alumina, sulfated zirconia, and mixtures thereof. In some embodiments, the solid acid catalyst is an amorphous silica-alumina catalyst comprising, consisting essentially of or consisting of amorphous silica-alumina. In some embodiments, the amorphous silica-alumina catalyst is substantially free or free of any metals or metal compounds loaded on the amorphous silica-alumina. In some embodiments, the amount of metal or metal compound loaded on the amorphous silica-alumina is no more than 3%, or no more than 2%, or no more than 1%, or no more than 0.5%, or no more than 0.2%, or no more than 0.1%, or no more than 0.05%, or no more than 0.02%, or no more than 0.01%, based on the weight of the amorphous silica-alumina. In some embodiments, the solid acid catalyst comprises at least 50%, or at least 60%, or at least 70%, or at least 80%, or at least 90%, or at least 95%, or at least 98%, or at least 99% amorphous silica-alumina, based on the total weight of the solid acid catalyst. In some embodiments, the silica-alumina (e.g, amorphous silica-alumina) has Si:Al (silicon:aluminum) mole ratio of no more than 20, or no more than 15, or no more than 10, or no more than 9, or no more than 8, or no more than 7, or no more than 6, or no more than 5, or no more than 4, or no more than 3, or no more than 2, or no more than 1. In some embodiments, the silica-alumina (e.g, amorphous silica-alumina) has Si:Al mole ratio of at least 0.01, or at least 0.05, or at least 0.1, or at least 0.2, or at least 0.3, or at least 0.4, or at least 0.5.

In some embodiments, the hydrogenation catalyst is a heterogeneous catalyst. Examples of hydrogenation catalyst include one or more nobel metals (e.g., Pt, Pd, Ru) loaded on a support (i.e., supported nobel metal catalyst), that is, the hydrogenation catalyst comprises, consists essentially of or consists of one or more nobel metals loaded on a support. Preferred nobel metal is Pt and/or Pd. Examples of hydrogenation catalyst also include one or more non-nobel metals loaded on a support. Such non-nobel metal is selected from the group consisting of cobalt, nickel, iron, and mixtures thereof. Examples of support include activated carbon, silica (SiO₂), calcium carbonate, barium sulfate, metal oxides, and mixtures thereof. Preferred support is selected from the group consisting of silica, metal oxides, and mixtures thereof. In some embodiments, the support comprises or is alumina (Al₂O₃). Examples of hydrogenation catalyst also include cobalt oxides, nickel oxides and iron oxides.

In some embodiments, the hydrogenation catalyst is selected from the group consisting of Pd/alumina, Pt/alumina, Ru/alumina, Pd/C, Pt/C, Ru/C, Pd/silica, Pt/silica, Ru/silica, and mixtures thereof. In some embodiments, the hydrogenation catalyst is selected from the group consisting of Pd/alumina, Pt/alumina, and mixtures thereof. In some embodiments, the hydrogenation catalyst comprises or is Pd/alumina. In some embodiments, the hydrogenation catalyst comprises or is Pt/alumina. The term “X/Y”, as used herein with respect to a hydrogenation catalyst, means metal X loaded on support Y.

Typically, the content of the nobel metal is from 0.1 wt % to 15 wt % based on the total weight of the catalyst. In some embodiments, the content of the nobel metal is from 0.2 wt % to 10 wt %, or from 0.5 wt % to 10 wt %, or from 1 wt % to 10 wt %, based on the total weight of the catalyst. In some embodiments, the upper limit of the content of the nobel metal is 15 wt %, or 14 wt %, or 13 wt %, or 12 wt %, or 11 wt %, or 10 wt %, or 9 wt %, or 8 wt %, or 7 wt %, based on the total weight of the catalyst. In some embodiments, the lower limit of the content of the nobel metal is 0.001 wt %, or 0.01 wt %, or 0.1 wt %, or 0.2 wt %, or 0.4 wt %, or 0.5 wt %, or 0.8 wt %, or 1 wt %, or 1.2 wt %, or 1.4 wt %, or 1.6 wt %, or 1.8 wt %, or 2 wt %, based on the total weight of the catalyst.

In some embodiments, both the solid acid catalyst and the hydrogenation catalyst are in powder form and are mixed uniformly to form the catalyst mixture which is then pelletized and sieved to the desired particle size. In some embodiments, a suitable binder can be used with the catalyst mixture to form particles with proper strength. In some embodiments, the catalyst mixture is in the form of particles and the particle size is from about 50 μm to about 1000 μm, or from about 100 μm to about 800 μm, or from about 150 μm to about 600 μm, or from about 200 μm to about 400 μm. In some embodiments, at least 50% of the particles, or at least 60% of the particles, or at least 70% of the particles, or at least 75% of the particles, or at least 80% of the particles, or at least 85% of the particles, or at least 90% of the particles, or at least 95% of the particles, or at least 98% of the particles, by number of the particles, comprises, consists essentially of or consists of both the solid acid catalyst and the hydrogenation catalyst. In some embodiments, essentially each or each particles of the catalyst mixture comprises, consists essentially of or consists of both the solid acid catalyst and the hydrogenation catalyst.

In some embodiments, the solid acid catalyst is an amorphous silica-alumina catalyst and the hydrogenation catalyst is a supported nobel metal catalyst (e.g., Pd/alumina). The weight ratio of silica-alumina to nobel metal is from about 1000:1 to about 5000:1, or from about 1600:1 to about 4500:1, or from about 2000:1 to about 4500:1, or from about 2000:1 to about 4000:1. In some embodiments, the upper limit of the weight ratio is 6500, 6000, 5500, 5000, 4800, 4500, 4200, 4000, 3800, 3500, 3200, or 3000. In some embodiments, the lower limit of the weight ratio is 500, 800, 1000, 1200, 1500, 1600, 1800, 2000, 2100, 2200, 2300, or 2400.

In some embodiments, the process of this disclosure is conducted at a temperature (reaction temperature, or temperature in the reaction zone) of from about 150° C. to about 400° C., or from about 200° C. to about 300° C., or from about 230° C. to about 270° C., or from about 220° C. to about 260° C. In some embodiments, the reaction temperature is at least 120° C., or at least 130° C., or at least 140° C., or at least 150° C., or at least 160° C., or at least 170° C., or at least 180° C., or at least 190° C., or at least 200° C., or at least 210° C., or at least 220° C. In some embodiments, the reaction temperature is no more than 400° C., or no more than 380° C., or no more than 360° C., or no more than 340° C., or no more than 320° C., or no more than 300° C., or no more than 280° C., or no more than 270° C., or no more than 260° C.

The process of this disclosure can be conducted under a pressure (reaction pressure, or pressure in the reaction zone) of no more than about 25 bar, or no more than about 20 bar, or no more than about 15 bar, or no more than about 10 bar, or no more than about 5 bar, or no more than about 4 bar, or no more than about 3 bar, or no more than about 2 bar, or no more than about 1.5 bar. In some embodiments, the reaction pressure is at least about 0.01 bar.

The contact time in the process of this disclosure can range from about 0.25 hour to about 2 hours, or from about 0.4 hour to about 1.5 hours, or from about 0.5 hour to about 1 hour. In some embodiments, the contact time is at least 0.05 hour, or at least 0.1 hour, or at least 0.15 hour, or at least 0.2 hour, or at least 0.25 hour, or at least 0.3 hour, or at least 0.4 hour, or at least 0.5 hour. In some embodiments, the contact time is no more than 4 hours, or no more than 3 hours, or no more than 2.5 hours, or no more than 2 hours, or no more than 1.5 hours, or no more than 1 hour.

The process of this disclosure produces a product mixture comprising BCI. In some embodiments, the product mixture also comprises hydrogenated MCDDK, MDIENE, position isomers of BCI, and/or saturated BCI as byproducts. The desired product BCI can be separated and recovered by methods known in the art such as distillation. In some embodiments, the yield of BCI is at least 60%, or at least 65%, or at least 70%, or at least 75%, or at least 80%. The process of this disclosure is a one-step process. The term “one-step”, as used herein with respect to a process, means that the process produces BCI directly without separating or purifying any reaction intermediates generated during the process.

Many aspects and embodiments have been described above and are merely exemplary and not limiting. After reading this specification, skilled artisans appreciate that other aspects and embodiments are possible without departing from the scope of the invention.

### EXAMPLES

The concepts described herein will be further described in the following examples, which do not limit the scope of the invention described in the claims.

### Fixed Catalyst Bed Preparation

The solid acid catalyst and the hydrogenation catalyst were mixed in powder form and then pelletized and sieved to the particle size of 200-400 μm. The particles of the catalyst mixture were then mixed with carborundum (SiC, as diluent) particles at 1:1 volume ratio. The resulting mixture was loaded into a vertical tubular reactor to form a fixed catalyst bed. An additional amount of SiC was loaded into the fixed bed reactor to form a SiC layer on top of the catalyst bed. The reactor was then heated to 120° C. for 6 hours with nitrogen flow through to dry and activate the solid acid catalyst.

### General Procedure

MCDDK and hydrogen were fed into a pre-heater to be mixed and preheated at 200° C. to form a gaseous mixture. The MCDDK/hydrogen gaseous mixture was then fed into the top of the fixed bed reactor and passed through the reactor in a downflow mode. The product mixture effluent from the bottom of the reactor was collected in a gas-liquid trap to be cooled and gas (e.g., hydrogen) was separated from the liquid product mixture. Samples of the liquid product mixture were taken for GC-MS analysis.

### Legend

SiAl is an amorphous silica-alumina catalyst (solid acid catalyst). The amorphous silica-alumina catalyst is the DAVICAT® 0702 catalyst obtained from W.R. Grace & Co.

Pd/C is palladium loaded on activated carbon support (hydrogenation catalyst). The Pd content is 5 wt % based on the total weight of the catalyst. The catalyst was obtained from Clariant.

Pd/Al₂O₃ is palladium loaded on alumina support (hydrogenation catalyst). The Pd content is 5 wt % based on the total weight of the catalyst. The catalyst was obtained from Johnson Matthey.

Ru/Al₂O₃ is ruthenium loaded on alumina support (hydrogenation catalyst). The Ru content is 5 wt % based on the total weight of the catalyst. The catalyst was obtained from Johnson Matthey.

Pt/Al₂O₃ is platinum loaded on alumina support (hydrogenation catalyst). The Pt content is 5 wt % based on the total weight of the catalyst. The catalyst was obtained from Merk.

### Example 1 (Comparative)

Only the amorphous silica-alumina was used as the catalyst. No hydrogenation catalyst was used in this Example. Reaction conditions and results are shown in Table 1.

TABLE 1

| | | | | Selectivity (%) | | |
|---|---|---|---|---|---|---|
| T | CT | H₂/MCDDK | Conversion | BCI + | | |
| (° C.) | (hrs) | (mole ratio) | (%) | Iso | MDIENE | Others |
| 200 | 0.50 | 40 | 96.8 | 6.4 | 18.9 | 74.7 |
| 210 | 0.54 | 40 | 97.2 | 0.0 | 47.8 | 52.2 |
| 250 | 0.57 | 40 | 97.3 | 3.3 | 56.7 | 40.0 |
| 300 | 0.56 | 40 | 99.1 | 11.5 | 54.3 | 34.2 |

Note: In Tables, T means reaction temperature in Celsius; CT means contact time in hours; H₂/MCDDK means the mole ratio of hydrogen to MCDDK fed into the reaction zone; Conversion means the conversion rate of MCDDK; and “BCI+Iso” means BCI and its position isomers.

### Example 2

Various catalyst mixtures were tested for the process of this disclosure. Reaction temperature was 200° C. Contact time was 0.5 hour. The mole ratio of hydrogen to MCDDK fed into the reaction zone was 40. Other reaction conditions and results are shown in Table 2.

TABLE 2

| | | Con- | | | |
|---|---|---|---|---|---|
| Catalyst | SiAl/Metal | version | Selectivity (%) | | |
| Mixture | (wt ratio) | (%) | BCI | MDIENE | Others |
| SiAl + Pd/C | 480 | 98.2 | 2.0 | 60.2 | 37.8 |
| SiAl + Pd/Al₂O₃ | 4980 | 98.4 | 37.1 | 33.9 | 29.0 |
| SiAl + Pd/Al₂O₃ | 2480 | 99.1 | 47.5 | 17.2 | 35.3 |
| SiAl + Ru/Al₂O₃ | 2480 | 95.0 | 20.3 | 57.4 | 22.3 |
| SiAl + Pt/Al₂O₃ | 3820 | 99.8 | 38.7 | 10.9 | 50.4 |
| SIAl + Pd/C | 75 | 98.0 | 0.6 | 60.0 | 39.4 |

Note: In Tables, SiAl/Metal means the weight ratio of silica-alumina to nobel metal.

### Example 3

In Example 3, the contact time effect was tested for the process of this disclosure. The catalyst mixture used in the process was a mixture of the amorphous silica-alumina catalyst and the Pd/Al₂O₃ catalyst. The weight ratio of silica-alumina to nobel metal (Pd) was 2480. Reaction temperature was 200° C. Other reaction conditions and results are shown in Table 3.

TABLE 3

| | H₂/MCDDK | Conversion | Selectivity (%) | | |
|---|---|---|---|---|---|
| CT (hrs) | (mole ratio) | (%) | BCI | MDIENE | Others |
| 0.5 | 40 | 97.2 | 23.7 | 18.8 | 57.5 |
| 1 | 40 | 99.1 | 74.3 | 0.9 | 24.8 |

### Example 4

In Example 4, the reaction temperature effect was tested for the process of this disclosure. The catalyst mixture used in the process was a mixture of the amorphous silica-alumina catalyst and the Pd/Al₂O₃ catalyst. The weight ratio of silica-alumina to nobel metal (Pd) was 2480. Other reaction conditions and results are shown in Table 4.

TABLE 4

| | CT | H₂/MCDDK | Conversion | Selectivity (%) | | |
|---|---|---|---|---|---|---|
| T (° C.) | (hrs) | (mole ratio) | (%) | BCI | MDIENE | Others |
| 200 | 1 | 40 | 99.1 | 74.3 | 0.9 | 24.8 |
| 250 | 1 | 40 | 99.1 | 79.4 | 0.0 | 20.6 |

### Example 5

In Example 5, the H₂/MCDDK mole ratio effect was tested for the process of this disclosure. The catalyst mixture used in the process was a mixture of the amorphous silica-alumina catalyst and the Pd/Al₂O₃ catalyst. The weight ratio of silica-alumina to nobel metal (Pd) was 3820. Reaction temperature was 250° C. Other reaction conditions and results are shown in Table 5.

TABLE 5

| CT | H₂/MCDDK | Conversion | Selectivity (%) | | |
|---|---|---|---|---|---|
| (hrs) | (mole ratio) | (%) | BCI | MDIENE | Others |
| 1.0 | 20 | 99.0 | 81.1 | 0.0 | 18.9 |
| 1.0 | 40 | 99.1 | 82.0 | 0.9 | 17.1 |

### Example 6

Various solid acid catalysts were tested for the process of this disclosure. Reaction temperature was 250° C. Contact time was 1 hour. The mole ratio of hydrogen to MCDDK fed into the reaction zone was 20. The weight ratio of solid acid catalyst to nobel metal (Pd) was 3820. Other reaction conditions and results are shown in Table 6.

TABLE 6

| | Conversion | Selectivity (%) | | |
|---|---|---|---|---|
| Catalyst Mixture | (%) | BCI | MDIENE | Others |
| γ-Al₂O₃ + Pd/Al₂O₃ | 98.1 | 40.0 | 0.5 | 59.5 |
| CBV-400 + Pd/Al₂O₃ | 94.8 | 24.9 | 10.1 | 66.7 |
| SiAl + Pd/Al₂O₃ | 99.1 | 81.1 | 0.0 | 18.9 |
| Sulfated zirconia + Pd/Al₂O₃ | 37.0 | 0.5 | 0.1 | 99.4 |

Note: γ-Al₂O₃ is a commercial gamma alumina from Sasol (Puralox Sba-200). CBV-400 is a commercial 12 ring member USY zeolite from Zeolyst.

### Example 7

Catalyst mixture of SiAl and Pd/Al₂O₃ was compared with Pd/SiAl catalyst alone. Pd/SiAl is palladium loaded on SiAl support. The Pd content is 0.025 wt % based on the total weight of the catalyst. Reaction temperature was 250° C. Contact time was 1 hour. The mole ratio of hydrogen to MCDDK fed into the reaction zone was 20. In the catalyst mixture of SiAl and Pd/Al₂O₃, the weight ratio of SiAl to nobel metal (Pd) was 3820. Other reaction conditions and results are shown in Table 7.

TABLE 7

| | | Selectivity (%) | | |
|---|---|---|---|---|
| Catalyst | Conversion (%) | BCI | MDIENE | Others |
| SiAl + Pd/Al₂O₃ | 99.1 | 81.1 | 0.0 | 18.9 |
| Pd/SiAl | 99.5 | 24.3 | 0.0 | 75.7 |

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination.

1. A process for making BCI, comprising: contacting a starting material comprising MCDDK with hydrogen in the presence of a catalyst mixture in a reaction zone to produce a product mixture comprising BCI, wherein the catalyst mixture comprises a solid acid catalyst and a hydrogenation catalyst.

2. The process of claim 1, wherein the mole ratio of hydrogen to MCDDK fed into the reaction zone is from about 5:1 to about 100:1.

3. The process as in claim 1, wherein the solid acid catalyst is selected from the group consisting of alumina, silica-alumina, zeolite, silico-alumino-phosphate, aluminophosphate, sulfated zirconia, zirconia, zinc oxide, and mixtures thereof.

4. The process as in claim 1, wherein the solid acid catalyst is an amorphous silica-alumina catalyst.

5. The process as in claim 1, wherein the hydrogenation catalyst is a supported nobel metal catalyst.

6. The process of claim 5, wherein the support is alumina.

7. The process as in claim 1, wherein the hydrogenation catalyst is Pd/alumina and/or Pt/alumina.

8. The process as in claim 1, wherein the catalyst mixture is in a form of particles, and at least 50% of the particles comprise both the solid acid catalyst and the hydrogenation catalyst.

9. The process as in claim 1, wherein the solid acid catalyst is an amorphous silica-alumina catalyst, the hydrogenation catalyst is a supported nobel metal catalyst, and the weight ratio of silica-alumina to nobel metal is from about 1000:1 to about 4500:1.

10. The process as in claim 1, wherein reaction temperature is from about 150° C. to about 400° C.

11. The process as in claim 1, wherein yield of BCI is at least about 75%.
